(19) United States
(12) Patent Application Publication
(10) Pub. No.: US 2006/0293574 A1
(43) Pub. Date: Dec. 28, 2006

(54) Separating oximeter signal components based on color

(76) Inventor: Mark A. Norris, Louisville, CO (US)

Correspondence Address: MARSH, FISCHMANN & BREYFOGLE LLP, 3151 SOUTH VAUGHN WAY, SUITE 411, AURORA, CO, 80014, US

(21) Appl. No.: 11/251,238

(22) Filed: Oct. 14, 2005

Related U.S. Application Data
(60) Provisional application No. 60/694,760, filed on Jun. 28, 2005.

(51) Int. Cl. A61B 5/00

(52) U.S. Cl. 600/323

(57) ABSTRACT

A component of interest of an oximeter detector signal is distinguished from an interfering component based on a color difference between the components. The component of interest may be a pulsatile signal component, a baseline signal component such as a respiratory signal, or an artifact signal. The color difference between the component of interest and the interfering component is reflected in corresponding mixing ratios with respect to multiple optical channels of the pulse oximeter. The component of interest is separated from the interfering component by mathematical decomposition using the mixing ratios. In this manner, signal components can be isolated substantially free from frequency dependent filtering.

### CROSS REFERENCE TO RELATED APPLICATIONS

This application claims priority from U.S. Provisional Application Ser. No. 60/694,760 entitled “SEPARATING OXIMETER SIGNAL COMPONENTS BASED ON COLOR”, filed on Jun. 28, 2005, the entirety of which is incorporated herein by reference.

### FIELD OF THE INVENTION

The present invention relates generally to pulse oximetry and, in particular, to signal processing techniques and associated structure for distinguishing a component of interest of an oximeter detector signal from an interfering component.

### BACKGROUND OF THE INVENTION

Pulse oximeters are used to determine various types of physiological information for a patient, based on transmission/absorption characteristics of light transmitted through or reflected from a patient's tissue. In particular, pulse oximeters generally include a probe for attaching to a patient's appendage such as a finger, earlobe or nasal septum, or another location, particularly in the case of reflective oximeters. The probe is used to transmit pulsed optical signals of at least two wavelengths, typically red and infrared, to the patient's tissue. The transmitted signals are received by a detector that provides an analog electrical output signal representative of the received optical signals. By processing the electrical signal and analyzing signal values for one or more of the wavelengths, information can be obtained regarding blood oxygen saturation and/or other parameter values such as pulse rate, respiration rate or other blood pressure/blood volume related values.

The algorithms for determining such values are normally implemented in a digital processing unit. Accordingly, one or more analog-to-digital (A/D) converters are generally interposed between the detector and the digital processing unit. Additionally, the detector signal is generally demodulated and demultiplexed by signal processing modules. Demodulation involves separating the physiological signal of interest (generally including a more rapidly changing AC portion including a plethysmographic waveform and an optically based “DC” offset due to slowly changing absorption values associated with non-pulsatile tissue absorption) from a carrier waveform associated with the flashing optical sources. Demultiplexing involves separating the different wavelength signals associated with the different signal sources. That is, because certain parameters such as blood oxygen saturation are calculated based on differential absorption values for different transmitted optical signal wavelengths, multiple channels are typically employed in the pulse oximeter and the detected signal is generally separated, or demultiplexed, into at least two different channel signals. Typically, demodulation and demultiplexing have been implemented in analog circuitry operatively disposed between the optical signal detector and the analog-to-digital converter(s), but can be digitally implemented. As a result of such processing, one or more AC signals are obtained for analysis to yield the desired physiological information.

These AC signals may include multiple signal components including components of physiological as well as nonphysiological origin. Such physiological components may include a pulsatile component corresponding to cardiac activity, a respiration component corresponding to respiratory activity and a Mayer Wave component which, while not fully understood, appears to have a relation to the vaso motor center. The nonphysiological components may be associated with patient motion (patient induced or otherwise), electronic noise, optical noise (e.g., from ambient light) or other artifact. Generally, the nonphysiological components are not of interest in connection with obtaining the desired physiological information.

However, various physiological components may be of interest. In this regard, the pulsatile component is typically processed to yield pulse rate and oxygen saturation values as well as to display the pulsatile waveform. The respiration component can be processed to provide a breathing rate for the patient which, particularly when considered in conjunction with arterial oxygen saturation, may be of interest to a physician. The Mayer Wave also has significance for diagnostic and patient monitoring purposes. In particular, the amplitude and frequency of the Mayer Wave are seen to change in connection with hypertension, sudden cardiac death, ventricular tachycardia, coronary artery disease, myocardial infarction, heart failure, diabetes, and autonomic neuropathy and after heart transplantation. Accordingly, it may be desired to isolate or distinguish any one of various components in the AC signal under analysis.

Various types of filtering techniques have been proposed or implemented to distinguish a component of interest from other components. The pulsatile component has often been the component of interest in this regard. Some approaches have attempted to selectively pass one or more frequency bands associated with the pulsatile component. Other approaches have attempted to selectively notch or block frequency bands associated with an interfering component. In the case of distinguishing the pulsatile component from the respiratory or Mayer Wave components, high pass or low pass filters have been proposed as the pulsatile signal is generally expected to have a higher frequency than those components.

Such frequency dependent filtering techniques, however, have certain limitations. First, the expected or actual frequency ranges of these components may overlap, complicating efforts to isolate a component based on frequency dependent filtering. Even where the primary frequencies of these components are different, harmonics of one component may interfere with another component, thereby hampering certain processing techniques. For example, such harmonic interference may be problematic in identifying a pulsatile signal in cases of low perfusion. Moreover, frequency dependent filtering may result in the loss of useful information. Accordingly, alternate techniques continue to be investigated for distinguishing components of oximeter signals.

### SUMMARY OF THE INVENTION

The present invention is directed to distinguishing signal components, e.g., isolating one or more signal components of interest, of a medical instrument such as a pulse oximeter, based on a mixing ratio or color analysis. Such an analysis allows the composite signal to be decomposed to yield the signal or signals of interest, preferably without requiring frequency dependent filtering. In this manner, components can be effectively distinguished even where the components may have overlapping frequencies. Moreover, potential interference associated with harmonics of a filtered frequency is substantially avoided. Loss of useful information can also be avoided or minimized, as it is unnecessary to block frequency bands.

The present inventor has recognized that signal components may have different colors that allow for distinguishing of such components. For example, in the case of pulse oximeters, it is expected that the pulsatile signal will generally be associated with arterial blood which characteristically has a bright red color. This color results in different levels of attenuation of the different channels of optical signals (typically at least including red and infrared channels) employed by the pulse oximeter. Other physiological components such as a respiratory or Mayer Wave component may be significantly associated with venous blood and tissue resulting in a different color characteristic. Thus, for example, the pulsatile component and respiratory component may be present in the red and infrared channel signals but with different mixing ratios. In this regard, certain motion artifact may be evenly mixed between red and infrared channels and other motion artifact may have different mix. Thus, in a variety of cases, different components have different color characteristics.

In accordance with the present invention, these color characteristics can be used to separate a detector signal into components. In one implementation, the detector signal is mathematically modeled as follows:

$${S}_{1} = {s}_{11} + {s}_{12} + … + {s}_{1 m}$$

$${S}_{2} = {s}_{21} + {s}_{22} + … + {s}_{2 m} \\ \cdot \\ \cdot \\ \cdot \\ {S}_{n} = {s}_{n 1} + {s}_{n 2} + … + {s}_{nm}$$

where

S_{1 . . . n}=the signal for channels 1 . . . n; and

s_{11 . . .} sₙₘ=the m signal components for the signals of channels 1 . . . n. Further, a given component as between different channels is defined by a mixing ratio for that component/channel combination. Thus, the case of an oximeter detector signal including a baseline component (such as a respiratory wave) and a pulsatile component in each of red and infrared channels, may be modeled as follows;

S_{red}=ₛ_{pred}+s_{bred}

Sᵢᵣ=sₚᵢᵣ+s_{bir where}

s_{pred}=the pulsatile component of the red channel signal S_{red};

s_{bred}=the baseline component of the channel signal;

sₚᵢᵣ=the pulsatile component of the infrared channel signal Sᵢᵣ; and

s_{bir}=the baseline component of the infrared channel signal. the mixing ratios are given by:

$${r}_{p} = \frac{{s}_{pred}}{{s}_{pir}}$$

$${r}_{b} = \frac{{s}_{bred}}{{s}_{pir}}$$

$$where$$

$${r}_{p} \neq {r}_{b}$$

It has been found that this mathematical model can be used to resolve each of the channel signals into its respective components, for example, yielding red and infrared pulsatile signals, substantially free of the baseline component, that can be used for pulserate and arterial oxygen saturation (SpO₂) calculations via conventional algorithms.

In accordance with one aspect of the present invention, a method and apparatus (collectively “utility”) is provided for distinguishing a desired component of an oximeter detector signal from another component. The utility involves receiving a detector signal including first and second components, distinguishing the first component of the detector signal from the second component based on a difference in color between the components, and using the first component to determine physiological information regarding a patient. For example, the first component may be a pulsatile component, a respiratory component, a Mayer Wave component, or other physiological component. The second component may comprise another of these physiological components or a nonphysiological component. The color of the signal components may be distinguished based on differing contributions of the respective components in channels corresponding to different optical spectral compositions. In this regard, the process for distinguishing the first component from the second component may involve applying a mathematical model for resolving each of the channel signals as the sum of the signal components where color related characteristics of the signal components can be used to solve for the desired first signal component in at least one of the channels. In this manner, the desired signal component can be distinguished despite potentially overlapping frequencies of the signal components and substantially without losing useful information due to frequency dependent filtering.

In accordance with another aspect of the present invention, a utility is provided for distinguishing a pulsatile component of an oximeter signal from another physiological component such as a respiratory component, Mayer Wave component or other baseline component. The utility involves: receiving a first channel signal of a pulse oximeter; resolving the first channel signal into pulsatile and baseline components based on an analysis relating to mixing ratios, with respect to the first and second channels, of each of the pulsatile and baseline components; selecting one of the pulsatile and baseline components; and processing the selected component to obtain physiological information regarding the patient. Depending on the specific implementation, one or both of the channel signals may be resolved into the pulsatile and baseline components. For example, in the case of determining a pulse rate, the pulsatile component of a single channel signal may be sufficient. On the other hand, in the case of determining an SpO₂ value, the pulsatile component may be desired for both of the channels.

### BRIEF DESCRIPTION OF THE DRAWINGS

For a more complete understanding of the present invention and further advantages, reference is now made to the following detailed description taken in conjunction with the drawings, in which:

FIG. 1 is a block diagram illustrating a pulse oximeter in accordance with the present invention;

FIGS. 2A-2C illustrate a process for resolving detector signals into components in accordance with the present invention;

FIG. 3 is a schematic diagram illustrating a system for implementing a color based filter in accordance with the present invention; and

FIG. 4 is a flow chart illustrating a process for decomposing a detector signal into each constituent component based on a color analysis in accordance with the present invention.

### DETAILED DESCRIPTION

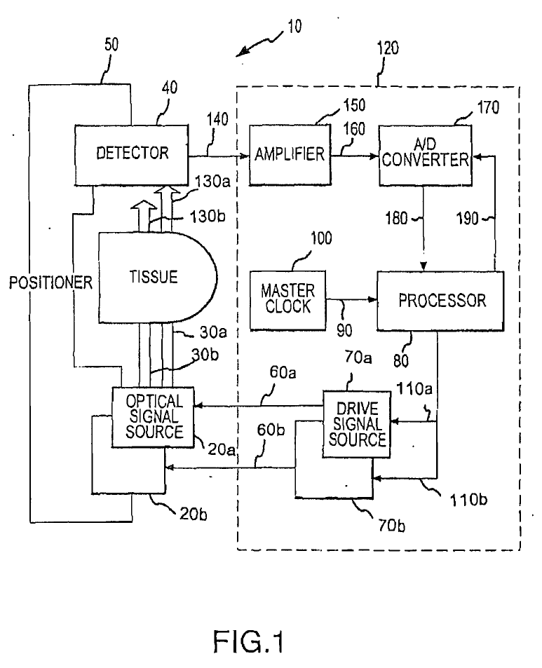

Referring now to FIG. 1, there is shown a block diagram of one embodiment of a pulse oximeter 10 in which a color filter in accordance with the present invention may be implemented. The pulse oximeter 10 is configured for use in determining the pulse rate of a patient as well as one or more blood analyte levels in the patient, such as an SpO₂ level. It should be appreciated that a color filter in accordance with the present invention may be implemented in pulse oximeters that are configured differently from the pulse oximeter depicted in FIG. 1 as well as in other environments wherein plethysmographic signals are processed in order to obtain desired information relating to patient physiological conditions from the plethysmographic signals.

The illustrated pulse oximeter 10 includes a pair of optical signal sources 20a, 20b for emitting a corresponding pair of light signals 30a, 30b centered at different predetermined center wavelengths λ₁, λ₂ through a suitable tissue site of a patient and on to a detector 40 (e.g., a photo-sensitive diode). It will be appreciated that the signals may be reflected from the tissue rather than transmitted through the tissue in accordance with the present invention.

The optical signal sources 20a, 20b and detector 40 may be included in a positioning device 50, e.g., associated with a probe, to facilitate alignment of the light signals 30a, 30b with the detector 40. For example, the positioning device 50 may be of clip-type or flexible strip configuration adapted for selective attachment to a suitable patient tissue site (e.g., a finger, an ear lobe, a foot, or the nose of the patient). The center wavelengths λ₁, λ₂ required depend upon the blood analyte level to be determined. For example, in order to determine an SpO₂ level, λ₁ may be in the red wavelength range and λ₂ may be in the infrared wavelength range. It should be appreciated that the pulse oximeter 10 may be readily implemented with more optical signal sources (e.g., four) depending, for example, upon the number of different blood analyte levels to be measured.

The optical signal sources 20a, 20b are activated by a corresponding plurality of drive signals 60a, 60b to emit the light signals 30a, 30b. The drive signals 60a, 60b are supplied to the optical signal sources 20a, 20b by a corresponding plurality of drive signal sources 70a, 70b. The drive signal sources 70a, 70b may be connected with a digital processor 80, which is driven with a clock signal 90 from a master clock 100. The digital processor 80 may be programmed to define modulation waveforms, or drive patterns, for each of the optical signal sources 20a, 20b. More particularly, the digital processor 80 may provide separate digital trigger signals 110a, 110b to the drive signal sources 70a-b, which in turn generate the drive signals 60a, 60b. In this regard, the digital trigger signals 110a, 110b may be configured to provide for multiplexing of the drive signals 60a, 60b, and in turn the light signals 30a, 30b, in accordance with a multiplexing scheme (e.g., time division, frequency division, and/or code division multiplexing).

The drive signal sources 70a, 70b, processor 80 and clock 100 may all be housed in a monitor unit 120. While the illustrated embodiment shows the optical signal sources 20a, 20b physically interconnected with the positioning device 50 (e.g., mounted within the positioning device 50 or mounted within a connector end of a cable that is selectively connectable with the positioning device 50), it should be appreciated that the optical signal sources 20a, 20b may also be disposed within the monitor unit 120. In the latter case, the light signals 30a, 30b emitted from the optical signal sources 20a, 20b may be directed from the monitor unit 120 via one or more optical fibers to the positioning device 50 for transmission through the tissue site. Furthermore, the drive signal sources 70a, 70b may comprise a single drive signal generator unit that supplies each of the drive signals 60a, 60b to the optical signal sources 20a, 20b.

Transmitted light signals 130a, 130b (i.e., the portions of light signals 30a, 30b exiting the tissue) are detected by the detector 40. The detector 40 detects the intensities of the transmitted signals 130a, 130b and outputs a current signal 140 wherein the current level is indicative of the intensities of the transmitted signals 130a, 130b. As may be appreciated, the current signal 140 output by the detector 40 comprises a multiplexed signal in the sense that it is a composite signal including information about the intensity of each of the transmitted signals 130a, 130b. Depending upon the nature of the drive signals 60a, 60b, the current signal 140 may, for example, be time division multiplexed, wavelength division multiplexed, and/or code division multiplexed.

The current signal 140 is directed to an amplifier 150, which may be housed in the monitor unit 120 as is shown. As an alternative, the amplifier 150 may instead be included in a probe/cable unit that is selectively connectable with the monitor unit 120. The amplifier 150 converts the current signal 140 to a voltage signal 160 wherein a voltage level is indicative of the intensities of the transmitted signals 130a, 130b. The amplifier 150 may also be configured to filter the current signal 140 from the detector 40 to reduce noise and aliasing. By way of example, the amplifier 150 may include a bandpass filter to attenuate signal components outside of a predetermined frequency range encompassing modulation frequencies of the drive signals 60a, 60b.

Since the current signal 140 output by the detector 40 is a multiplexed signal, the voltage signal 160 is also a multiplexed signal, and thus, the voltage signal 160 is demultiplexed in order to obtain signal portions corresponding with the intensities of the transmitted light signals 130a, 130b. In this regard, the digital processor 80 may be provided with demodulation software for demultiplexing the voltage signal 160. In order for the digital processor 80 to demodulate the voltage signal 160, it is converted from analog to digital. Conversion of the analog voltage signal 160 is accomplished with an analog-to-digital (A/D) converter 170, which may also be included in the monitor unit 120. The A/D converter 170 receives the analog voltage signal 160 from the amplifier 150, samples the voltage signal 160, and converts the samples into a series of digital words 180 (e.g., eight, sixteen or thirty-two bit words), wherein each digital word is representative of the level of the voltage signal 160 (and hence the intensities of the transmitted light signals 130a, 130b) at a particular sample instance. In this regard, the A/D converter 170 preferably provides for sampling of the voltage signal 160 at a rate sufficient to provide for accurate tracking of the shape of the various signal portions comprising the analog voltage signal 160 being converted. For example, the A/D converter 170 may provide for a sampling frequency at least twice the frequency of the highest frequency drive signal 60a, 60b, and more preferably at an even greater sampling rate in order to more accurately represent the analog voltage signal.

The series of digital words 180 is provided by the A/D converter 170 to the processor 80 to be demultiplexed. More particularly, the processor 80 may periodically send an interrupt signal 190 (e.g., once per every eight, sixteen or thirty-two clock cycles) to the A/D converter 170 that causes the A/D converter 170 to transmit one digital word 180 to the processor 80. The demodulation software may then demultiplex the series of digital words 180 in accordance with an appropriate method (e.g., time, frequency and/or code) to obtain digital signal portions indicative of the intensities of each of the transmitted light signals 130a, 130b. In this regard, the demultiplexed digital signal portions comprise time domain plethysmographic signals corresponding to the center wavelengths λ₁, λ₂ (e.g., red and infrared) of the optical signal sources 20a, 20b. The red and infrared time domain plethysmographic signals may then be processed by the processor 80 to obtain desired patient physiological condition related information therefrom such as the patient's pulse rate and SpO₂ level.

In accordance with the present invention, the detector signal can be resolved into constituent components based on color. That is, as noted above, different components of the signal, including components of physiological origin and components of nonphysiological origins such as certain artifact, may have different colors. These different colors are associated with different mixing ratios relative to the different channels. Thus, a particular signal component may be substantially equally present in each channel, i.e., that component may be substantially white for purposes of the present analysis. By contrast, another signal component may have substantial color as reflected by a mixing ratio that is substantially different from one. Each of these components is therefore present in each of the channel signals. However, the relative contributions of the different components in the different channel signals will differ significantly. The above-noted mathematical model can be used to take advantage of these color characteristics to distinguish a particular component of interest based on color.

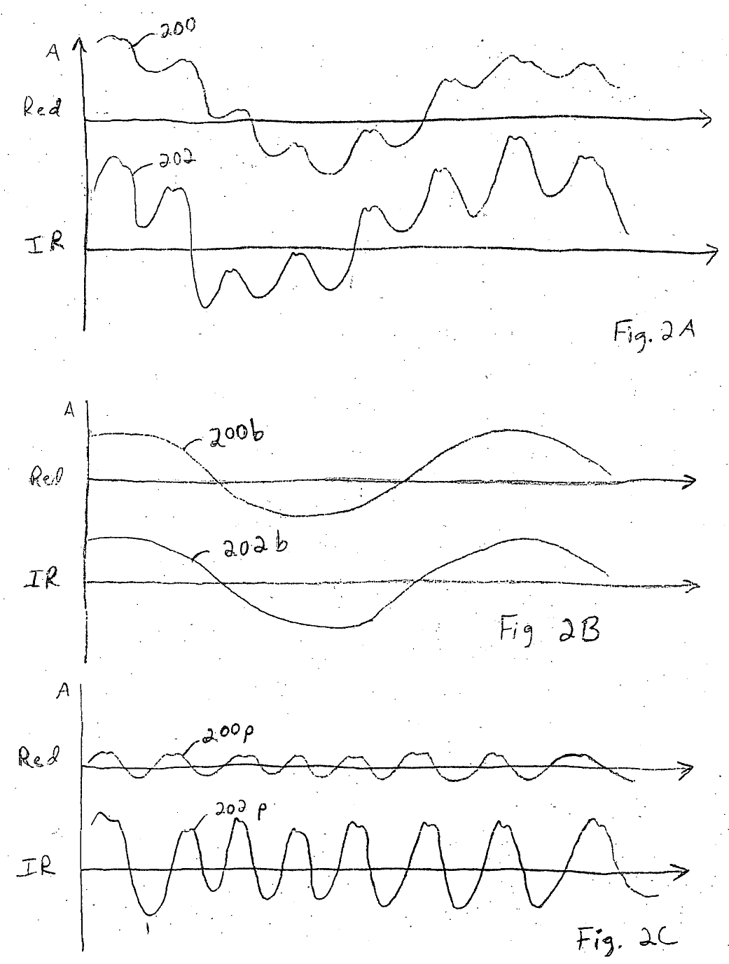

FIGS. 2A-2C illustrate an example of this process for resolving a detector signal into components based on color. In the illustrated example, a two-channel oximeter is employed including red and infrared source channels. In addition, the detector signal is assumed to be composed of a pulsatile component and a baseline component such as a respiratory wave component. It will be appreciated that an oximeter including more channels and a signal including more components may be involved in accordance with the present invention. Moreover, the color filter may be used in combination with other filters, including frequency dependent filters. For example, one or more components such as an artifact component and a physiological component (pulsatile, respiratory or Mayer Wave component), may be substantially eliminated prior to using the color filter to distinguish two remaining components.

Referring to FIG. 2A, an exemplary red channel signal 200 and infrared channel signal 202 are shown. For example, the signals 200 and 202 may represent the AC portions of a pulse oximeter detector signal after the signal has been digitized, demodulated (separated from the carrier signal), demultiplexed (separated into red and infrared channels), stripped of a DC offset, and otherwise processed. For purposes of the present description, it is assumed that the DC component offset has been stripped in a manner that preserved the pulsatile component as well a baseline signal as can generally be observed in FIG. 2A.

In accordance with the present invention, the red and infrared channel signals 200 and 202 can be resolved into the baseline and pulsatile components. The resulting components are illustrated in FIGS. 2B and 2C. Specifically, in FIG. 2B, the resulting baseline component of the red and infrared channels 200b and 202b are shown. In FIG. 2C, the pulsatile components 200p and 202p are shown. It will be observed in this example that the baseline signal is substantially evenly mixed as between the red and infrared channels whereas the pulsatile signal has a mixing ratio that departs significantly from unity. Indeed, the color reflected in the pulsatile signal corresponding to this mixing ratio is a fundamental property that enables SpO₂ to be determined by pulse oximeters.

The process for separating the components based on color involves use of the mathematical model as set forth above. Essentially, this is a problem of independent component analysis which has been used in other contexts, for example, to distinguish different speakers using a common microphone. In this case, pulse oximeters can provide at least two channels which allows for solving of two mixing ratios simultaneously which in turn enables separation of the channel signals 200 and 202 into the components 200b, 200p, 202b and 202p. The underlying math is well known. While this math is complex and somewhat nonlinear, it reduces in this case to solving a two by two matrix such that the implementation is not computationally intensive. That is, when the data points coming in on the red and infrared channel are multiplied by the correct values in the two by two array as determined by the mixing ratios, the signals are physically separated into their components according to the mixing ratio. The results are good in cases where there are two separate signals reflecting two separate recorded values of SpO_{2,} for example a pulsatile signal based on arterial blood and a respiratory signal significantly associated with venous blood and tissue or certain cases of substantially white artifact.

The associated independent components analysis module can be implemented in hardware or software. In one implementation, the module is implemented in the processor 80 of FIG. 1. Specifically, the processor receives the signals 200 and 202 after digitization, demodulation and demultiplexing as noted above. The signals 200 and 202 are then processed using the above-noted mathematical model to obtain the mixing ratios and populate the noted two by two matrix. The signals 200 and 202 are then processed using the matrix to separate the signals into the components 200b, 200p, 202b and 202p.

The desired components can then be used to obtain physiological information regarding the patient. For example, the pulsatile components 200p and 202p can be used to determine pulse rate and SpO₂ via conventional processes. For example, an FFT may be performed with respect to one of the signals 200p and 202p to obtain a spectrum where the fundamental frequency of the spectrum corresponds to pulse rate. In addition, the signals 200 and 202p may be analyzed in the time or frequency domain to calculate value correlated SpO₂. Alternatively, the value of SpO₂ may be correlated to the mixing ratios for the components 200p and 202p.

Additionally or alternatively, one or both of the components 200b or 202b may be processed to obtain physiological information. For example, in the case where the components 200b and 202b correspond to a respiratory signal, the signal may be processed to provide an output regarding the patient's breathing rate. For example, one of the signals 200b or 202b may be subjected to an FFT to provide a spectrum where the fundamental frequency of the spectrum corresponds to the patient's breathing rate. It will be appreciated that many other types of physiological information may be obtained from components such as the respiratory signal component or a Mayer Wave component.

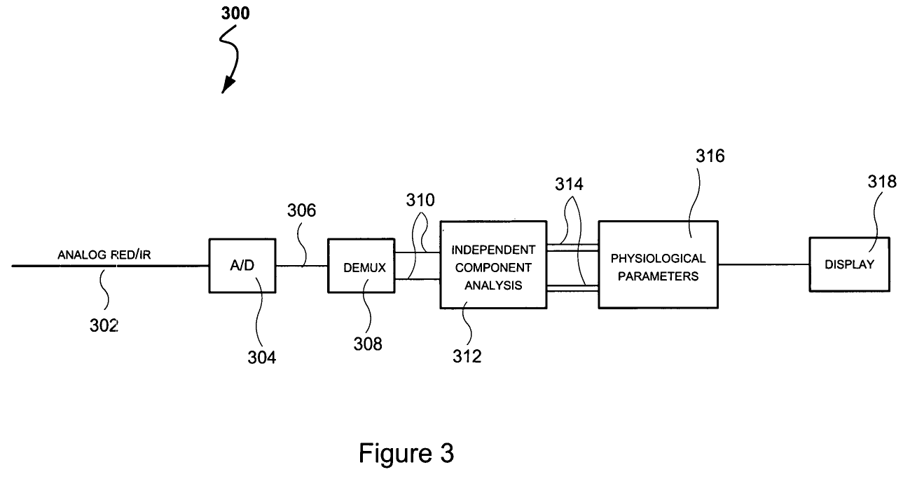

Referring to FIG. 3, a schematic diagram of a system 300 implementing an independent component analysis as discussed above is shown. The system 300 includes an analog-to-digital converter 304 that receives the analog red and infrared AC signals 302 and provides a digital signal 306 indicative thereof. The signal 306 is processed by a demultiplexer 308 to provide digital channel signals 310. These digital channel signals 310 are processed by the independent component analysis module 312 which implements the processing discussed above for separating the signals 310 into their respective components 314 based on color. One or more of the components 314 is then processed by a physiological parameter module 316 to obtain the desired physiological parameter (e.g., pulse rate, SpO₂, breathing rate, or blood pressure/volume information) which is then provided to the display 318 of the pulse oximeter.

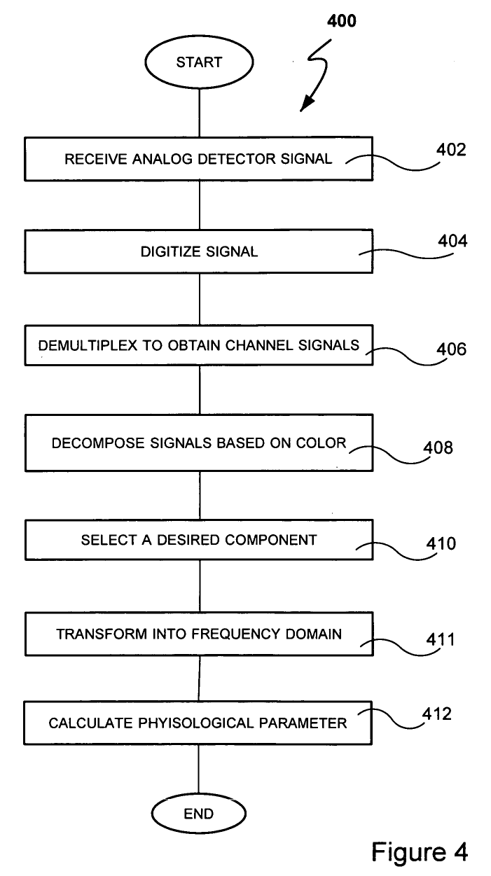

A corresponding process may be summarized by reference to the flow chart of FIG. 4. The illustrated process 400 is initiated by receiving (402) the analog detector signal. It will be appreciated that this signal may be amplified to provide an appropriate voltage signal, bandpass filtered to remove certain types of noise and other artifact, filtered to remove a DC offset and otherwise conditioned. The signal is then digitized (404) to provide an appropriate signal for processing by a digital signal processing unit. The digital signal is then demultiplexed (406) to obtain the separate channel signals. For example, in the case of code division multiplex signals, the step of demultiplexing may involve multiplication of the digital detector signal by appropriate demultiplexing vectors to yield the individual channel signals. The channel signals are then decomposed 408 into signal components based on color as described above. A desired component or components can then be selected (410) to yield the desired physiological information. For example, these components may be pulsatile components, respiratory wave components or Mayer Wave components. The desired components can be transformed into frequency domain spectra and then one or more of these spectra are then used to calculate (412) physiological parameter information for display by the pulse oximeter.

While various embodiments of the present invention have been described in detail, it is apparent that further modifications and adaptations of the invention will occur to those skilled in the art. However, it is to be expressly understood that such modifications and adaptations are within the spirit and scope of the present invention.

What is claimed:

1. A method for use in pulse oximetry, comprising the steps of:
receiving a first detector signal corresponding to a first optical signal channel of a pulse oximeter and a second detector signal corresponding to a second optical signal channel of said pulse oximeter;
resolving said first detector signal into first and second signal components based on an analysis related to mixing ratios, with respect to said first and second channels, of each of said first and second components; and
selecting one of said first and second components; and
processing said selected one of said first and second components to obtain physiological information regarding a patient.

2. A method as set forth in claim 1, wherein said step of resolving comprises determining a first mixing ratio of said first component with respect to said first and second channels and a second mixing ratio of said second component with respect to said first and second channels, wherein each of said first and second mixing ratios relates to a relative poser in said first and second channels of a respective one of said first and second components.

3. A method as set forth in claim 1, wherein said processing comprises determining one of a pulse rate and a respiration rate for said patient.

4. A method as set forth in claim 1, further comprising the step of resolving said second detector signal into said first and second components based on said analysis.

5. A method as set forth in claim 4, wherein said step of processing comprises using said selected one of said first and second components of each of said first and second detector signals to obtain said physiological information.

6. A method as set forth in claim 1, wherein said step of resolving comprises modeling each of said first and second detector signals as a sum of at least two components, determining mixing ratios for said at least two components with respect to at least two detector signals corresponding to at least two channels, and using said mixing ratios to resolve said first detector signal into said first and second components.

7. A method for use in pulse oximetry, comprising the steps of:
receiving a detector signal including a first component and a second component;
distinguishing said first component of said detector signal from said second component based on a difference in color between said first and second components; and
using said distinguished first component to determine physiological information regarding a patient.

8. A method as set forth in claim 7, wherein said first component is a pulsatile component ands said physiological information comprises one of pulse rate and blood oxygen saturation.

9. A method as set forth in claim 7, wherein said step of receiving comprises obtaining a digital signal corresponding to an optical signal of a pulse oximeter.

10. A method as set forth in claim 7, wherein said step of receiving further comprises receiving a second detector signal including said first and second components, wherein said first detector signal corresponds to a first channel of a pulse oximeter and said second detector signal corresponds to a second channel of said pulse oximeter.

11. A method as set forth in claim 10, wherein said separate distinguishing comprises determining a first mixing ratio of said first component with respect to said first and second channels and a second mixing ration of said second component with respect to said first and second channels.

12. A method as set forth in claim 10, wherein said step of distinguishing comprises modeling each of said first and second detector signals as a form of at least two components, determining mixing ratios for said at least two components with respect to at least two detector signals corresponding to at least two channels of said pulse oximeter, and using said mixing ratios to distinguish said first component of said detector signal from said second component.

13. A method for use in pulse oximetry, comprising the steps of:
receiving a detector signal including a first component and a second component;
mathematically decomposing said detector signal to isolate said first component from said detector signal by establishing a model of said detector signal as a combination of said first and second components and resolving said model to yield a representation of first components; and
processing said representation of said first component independent of said second component to provide physiological parameter information regarding a patient.

14. A method as set forth in claim 13, wherein said step of decomposing comprises obtaining said representation of said first component substantially free of frequency dependent filtering.

15. A method as set forth in claim 13, wherein said step of decomposing involves using a difference in color between said first and second components to isolate said first component.

16. A method as set forth in claim 15, wherein said difference in color is considered in relation to at least two optical signals of said pulse oximeter having different spectral contents.

17. A method as set forth in claim 16, wherein said step of decomposing involves determining mixing ratios with respect to measurements corresponding to said at least two optical signals.

18. A pulse oximetry apparatus, comprising:
a port for receiving first and second detector signals corresponding to first and second optical channels of said pulse oximeter, each of said first and second detector signals including first and second signal components; and
a processor for resolving said first detector signal into first and second components based on an analysis relating to mixing ratios with respect to said first and second channels of each of said first and second signal components, selecting one of said first and second components, and processing said selected one of said first and second components to obtain physiological information regarding said patient.

19. An apparatus as set forth in claim 18, wherein said processor is operative for performing said resolving by determining a first mixing ratio of said first component with respect to said first and second channels and a second mixing ratio of said second component with respect to said first and second channels, wherein each of said first and second mixing ratios relate to a relative power in said first and second channels of a respective one of said first and second components.

20. An apparatus as set forth in claim 18, wherein said processor is operative for performing said resolving by modeling each of said first and second detector signals as a sum of at least two components, determining mixing ratios for said at least two components with respect to at least two detector signals corresponding to at least two channels, and using said mixing ratios to resolve said first detector signal into said first and second components.

21. An apparatus as set forth in claim 18, wherein said processor is operative for determining one of a pulse rate and a blood oxygen saturation for said patient based on said selected one of said first and second components.
